(19) United States
(12) Patent Application Publication
(10) Pub. No.: US 2009/0306690 A1
(43) Pub. Date: Dec. 10, 2009

(54) ABRASIVE NOSE CONE WITH EXPANDABLE CUTTING AND SANDING REGION FOR ROTATIONAL ATHERECTOMY DEVICE

(75) Inventors: Jody Rivers, Elk River, MN (US); Ryan Welty, Blaine, MN (US); Matthew D. Cambronne, Moundsview, MN (US); David C. Franchino, Madison, WI (US); Kent Jeffrey Kallsen, Jefferson, WI (US); Stephen Latham, Sun Prairie, WI (US)

Correspondence Address: Altera Law Group, LLC, 220 S 6 St Suite 1700, Minneapolis, MN, 55402, US

(73) Assignee: CARDIOVASCULAR SYSTEMS, INC., St. Paul, MN (US)

(21) Appl. No.: 12/466,152

(22) Filed: May 14, 2009

Related U.S. Application Data
(60) Provisional application No. 61/059,028, filed on Jun. 5, 2008.

(51) Int. Cl. A61B 17/22

(52) U.S. Cl. 606/159

(57) ABSTRACT

An rotational atherectomy apparatus for abrading tissue, comprising: a flexible, elongated, rotatable drive shaft having a proximal end and a distal end opposite the proximal end; a nose cone operatively attached proximate the distal end of the drive shaft comprising a distal tapered section and a plurality of elongate, flexible members adjacent to the distal tapered section of the drive shaft, each member in the plurality being fixed at both a proximal end and a distal end opposite the proximal end; a proximal mount rotatable with the drive shaft and fixedly connected to the proximal ends of all the flexible members in the plurality; and a distal mount axially separated from the proximal mount and fixedly connected to the distal ends of all the flexible members in the plurality. When the axial separation of the proximal and distal mounts is reduced by pulling the distal tapered section proximally, each member in the plurality bows outward from the drive shaft and expands radially in an at least partially elliptical profile.

### CROSS-REFERENCE TO RELATED APPLICATIONS

The present application claims priority to provisional application No. 61/059028, filed on Jun. 5, 2008 under the title, “CUTTING AND SANDING RIBBON WISK”, the contents of which are incorporated by reference herein in its entirety.

### STATEMENT REGARDING FEDERALLY SPONSORED RESEARCH OR DEVELOPMENT

Not Applicable

### BACKGROUND OF THE INVENTION

1. Field of the Invention

The present invention is directed generally to expandable abrasive grinding and cutting heads for rotational atherectomy devices.

2. Description of the Related Art

Atherectomy is a non-surgical procedure to open blocked coronary arteries or vein grafts by using a device on the end of a catheter to cut or shave away atherosclerotic plaque (a deposit of fat and other substances that accumulate in the lining of the artery wall). For the purposes of this application, the term “abrading” is used to describe the grinding and/or scraping action of such an atherectomy head.

Atherectomy is performed to restore the flow of oxygen-rich blood to the heart, to relieve chest pain, and to prevent heart attacks. It may be done on patients with chest pain who have not responded to other medical therapy and on certain of those who are candidates for balloon angioplasty (a surgical procedure in which a balloon catheter is used to flatten plaque against an artery wall) or coronary artery bypass graft surgery. It is sometimes performed to remove plaque that has built up after a coronary artery bypass graft surgery.

Atherectomy uses a rotating shaver or other device placed on the end of a catheter to slice away or destroy plaque. At the beginning of the procedure, medications to control blood pressure, dilate the coronary arteries, and prevent blood clots are administered. The patient is awake but sedated. The catheter is inserted into an artery in the groin, leg, or arm, and threaded through the blood vessels into the blocked coronary artery. The cutting head is positioned against the plaque and activated, and the plaque is ground up or suctioned out.

The types of atherectomy are rotational, directional, and transluminal extraction. Rotational atherectomy uses a high speed rotating shaver to grind up plaque. Directional atherectomy was the first type approved, but is no longer commonly used; it scrapes plaque into an opening in one side of the catheter. Transluminal extraction coronary atherectomy uses a device that cuts plaque off vessel walls and vacuums it into a bottle. It is used to clear bypass grafts.

Several devices have been disclosed that perform rotational atherectomy. For instance, U.S. Pat. No. 5,360,432, issued on Nov. 1, 1994 to Leonid Shturman, and titled “Abrasive drive shaft device for directional rotational atherectomy” discloses an abrasive drive shaft atherectomy device for removing stenotic tissue from an artery, and is incorporated by reference herein in its entirety. The device includes a rotational atherectomy apparatus having a flexible, elongated drive shaft having a central lumen and a segment, near its distal end, coated with an abrasive material to define an abrasive segment.

Expandable atherectomy devices have been actively sought in order to gain the advantage of the small retracted position diameter during insertion and placement in the vasculature while allowing the device to achieve an expanded position comprising larger-than-retracted diameter during high-speed rotation.

Examples of such efforts are found in, e.g., U.S. Pat. No. 4,966,604 to Reiss; U.S. Pat. No. 5,030,201, issued on Jul. 9,1991 to Palestrant; U.S. Pat. No. 5,178,625, issued on Jan. 12, 1993 to Groshong; and U.S. Pat. No. 5,376,100, issued on Dec. 27, 1994 to Lefebvre. Each of these references are incorporated by reference herein in their entirety.

Collectively, the five references discussed above disclose atherectomy devices capable of moving cutting elements from a retracted position to an expanded position. In one aspect, the cutting elements include blades with and without abrasive thereon. Certain embodiments of these disclosed blades include sharp metal edges while others include flexible plastic cutting elements. Movement from retracted to expanded position may be achieved by mechanical manipulation by the operator using, for example, a central wire through the cutting element with a distal stop thereon which is slidable in response to pulling/pushing force applied by the operator. In some aspects, the distal cutting head is fixed to an axially slidable wire. A variation of this mechanism provides desmodromic wire(s) with a slidable tip and an axial motion stop to allow expansion (bowing) and retraction of the cutting wires. Alternatively, centrifugal force, for example, flexible plastic cutting elements or fibers, is sufficient to achieve the expanded position. The various designs achieve cutting and/or sanding by, for instance, rotating motion driven by a powered means.

Additional expandable cutting means are disclosed in the following references.

U.S. Pat. No. 7,291,146, issued on Nov. 6, 2007 to Steinke et al, discloses a radially expandable structure with electrosurgical energy delivery devices attached thereto, for example, electrodes, for engaging the atherosclerotic material.

U.S. Pat. No. 5,224,945, issued on Jul. 6, 1993 to Pannek discloses an expandable and compressible atherectomy cutter having a plurality of radially separated and axially joined blades. The distal ends of these blades are joined by a grommet which is slidable on a guide wire, while the proximal ends are fixed in position. Expansion of the blades is achieved by pulling on the guide wire, which includes a stop distal to the grommet.

U.S. Pat. No. 5,318,576, issued on Jun. 7, 1994 to Plassche Jr. et al, discloses a cutter of preferably ellipsoidal profile including a plurality of flexible segments. These flexible segments having cutting elements thereon, for example, cusps and mounds, distributed over the outer surface. The cutter is shortened axially to radially expand the flexible segments by use of a guide wire having a distal stop thereon.

U.S. Pat. No. 5,556,408, issued on Sep. 17, 1996 to Farhat discloses an expandable and compressible atherectomy cutter including a distal hub and a proximal hub joined by a plurality of resilient blades. The blades are shaped to describe a cutting radius which may be compressed.

U.S. Pat. No. 5,766,191, issued on Jun. 16, 1998 to Trerotola discloses a thrombolytic fragmentation device including a wire cage or basket attached to a rotational drive. The wire basket or cage is encased within a catheter and, when released therefrom, automatically conforms to the inner dimension of the vessel lumen. The wire basket or cage is defined in '704 as being made from three to six wires, made from a shape memory material such as nitinol, wherein the undeformed position is the expanded or bowed position. The wires may include abrasive thereon or have a cutting edge. The rotational speed disclosed is relatively low, with the highest disclosed speed being 5,000 rpm.

U.S. Pat. No. 6,800,083, issued on Oct. 5, 2004 to Hiblar et al, discloses a compressible atherectomy burr having one or more flexible abrasive disks foldable to be slidably received within a catheter. In another design, a resilient and flexible panel spirals outwardly, forming a generally cylindrical ablation surface.

In several of these references, members span a portion of the atherectomy head. These members are fixed at their proximal and distal ends, and are generally free in the portions between the proximal and distal ends. When the fixed proximal and distal ends of the members are brought toward each other, the members bow and expand radially outward. When rotated about the drive shaft, these radially expanded members can cut, scrap or grind at blockages that are larger in diameter than the rest diameter of the atherectomy head.

However, none of these references disclose a distal nose cone coated with abrasive, a proximal section of which is expandable. In this regard, the most distal taper of the nose cone may be used to open partially or completely occluded vessels, thereby creating a pilot hole. The remainder of the retracted cylindrical and low profile nose cone may then be gradually worked into the occlusion, whereupon the distal tapered section of the nose cone may be pulled back or distally, causing the cutting members to bow outwardly in an expanded position. Rotation of the nose cone in either the retracted or the expanded position facilitates opening of the occlusion.

### BRIEF SUMMARY OF THE INVENTION

One embodiment of the present invention comprises a rotational atherectomy apparatus for abrading tissue, comprising: a flexible, elongated, rotatable drive shaft having a proximal end and a distal end opposite the proximal end; a nose cone operatively attached proximate the distal end of the drive shaft comprising a distal tapered section and a plurality of elongate, flexible members adjacent to the distal tapered section of the drive shaft, each member in the plurality being fixed at both a proximal end and a distal end opposite the proximal end; a proximal mount rotatable with the drive shaft and fixedly connected to the proximal ends of all the flexible members in the plurality; and a distal mount axially separated from the proximal mount and fixedly connected to the distal ends of all the flexible members in the plurality. When the axial separation of the proximal and distal mounts is reduced by pulling the distal tapered section proximally, each member in the plurality bows outward from the drive shaft and expands radially. The axial separation of the proximal and distal mounts may be achieved by an actuator cable or tube through the inner diameter of the drive shaft and in operative communication with the distal tapered section. In the retracted position, the plurality of elongate, flexible members comprise a cylindrical profile while the distal tapered section comprises a proximal cylindrical profile with diameter equivalent to the retracted plurality of elongate, flexible members and a distal conical profile. In the expanded position, the plurality of elongate, flexible members comprise in one embodiment, an at least partially elliptical, symmetric, profile and in another embodiment, an eccentric or asymmetric profile, while the distal tapered section comprises a proximal cylindrical profile with diameter equivalent to the retracted plurality of elongate, flexible members and a distal conical profile. In other words, the distal tapered section's profile remains constant in both the retracted and expanded positions.

One embodiment of the present invention comprises a rotational atherectomy system for abrading tissue, comprising: a guide wire, a catheter having a lumen therethrough and capable of advancement/retraction over the guide wire; a flexible, elongated, rotatable drive shaft having a proximal end and a distal end opposite the proximal end; a nose cone operatively attached proximate the distal end of the drive shaft comprising a distal tapered section and a plurality of elongate, flexible members adjacent to the distal tapered section of the drive shaft, each member in the plurality being fixed at both a proximal end and a distal end opposite the proximal end; a proximal mount rotatable with the drive shaft and fixedly connected to the proximal ends of all the flexible members in the plurality; and a distal mount axially separated from the proximal mount and fixedly connected to the distal ends of all the flexible members in the plurality. When the axial separation of the proximal and distal mounts is reduced by pulling the distal tapered section proximally, each member in the plurality bows outward from the drive shaft and expands radially. The axial separation of the proximal and distal mounts may be achieved by an actuator cable or tube through the inner diameter of the drive shaft and in operative communication with the distal tapered section. In the retracted position, the plurality of elongate, flexible members comprise a cylindrical profile while the distal tapered section comprises a proximal cylindrical profile with diameter equivalent to the retracted plurality of elongate, flexible members and a distal conical profile. In the expanded position, the plurality of elongate, flexible members comprise in one embodiment, an elliptical, symmetric, profile and in another embodiment, an eccentric or asymmetric profile, while the distal tapered section comprises a proximal cylindrical profile with diameter equivalent to the retracted plurality of elongate, flexible members and a distal conical profile. In other words, the distal tapered section's profile remains constant in both the retracted and expanded positions. In addition, the system comprises means for rotating the drive shaft.

Another embodiment of the present invention is a method for abrading a blockage, comprising: advancing a guide wire through a vasculature of a patient to the blockage; advancing a catheter over the guide wire to the blockage; advancing a rotatable drive shaft comprising the inventive nose cone operatively attached proximate the distal end of the drive shaft within the catheter to the blockage; advancing the distal tapered section of the nose cone to the blockage and initiating rotating and/or axial movement of the nose cone; creating a pilot hole through the blockage if necessary; advancing the plurality of elongate, flexible members through the catheter proximate the blockage; and beyond a distal end of the catheter to the blockage, the members being fixedly connected at their proximal ends to a common proximal mount and being fixedly connected at their distal ends to a common distal mount, the proximal and distal mounts being axially separated, the proximal mount being rotatably coupled to the drive shaft; actuating the plurality of elongate, flexible members into an expanded position, wherein the plurality of flexible members bow radially outward from the drive shaft; rotating the drive shaft; abrading the blockage through repeated contact with the plurality of bowed flexible members; stopping the rotation of the drive shaft; returning the plurality of flexible members to the retracted position; withdrawing the plurality of flexible members into the catheter; withdrawing the system from the vasculature of the patient.

Alternate methods may comprise the flexible members being biased in the bowed expanded position so that the flexible members radially expand upon advancement out of the catheter and radially compress into a retracted position upon withdrawal into the catheter.

### BRIEF DESCRIPTION OF THE SEVERAL VIEWS OF THE DRAWINGS

FIG. 1 is a perspective drawing of one embodiment of a rotational atherectomy device.

FIG. 2 is a partial cutaway of one embodiment of a drive shaft with nose cone operatively attached proximate the distal end of the drive shaft and in a retracted position.

FIG. 3 is a partial cutaway of one embodiment of a drive shaft with nose cone operatively attached proximate the distal end of the drive shaft and in an expanded position.

### DETAILED DESCRIPTION OF THE INVENTION

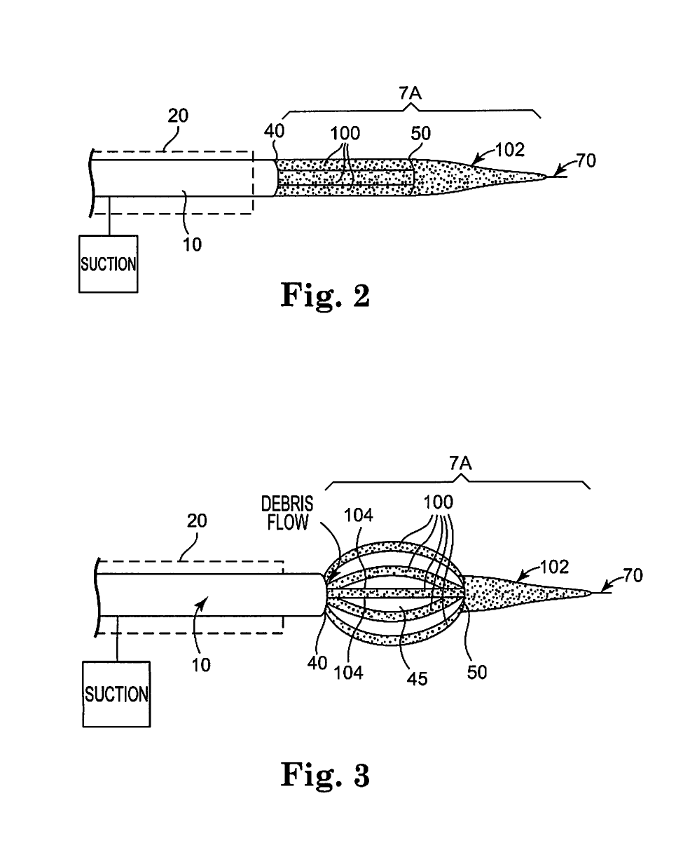

A rotational atherectomy device is disclosed, in which an abrasive nose cone is attached to a rotatable drive shaft. The abrasive head includes a distal tapered section and, proximal thereto, a plurality of elongate, flexible members, such as wires, that are attached at their proximal ends to a proximal mount, and are attached at their distal ends to a distal mount. The proximal and distal mounts and the distal tapered section are rotatably operatively attached to a drive shaft, and rotate with the drive shaft. The proximal and distal mounts are axially separated from each other. As the axial spacing decreases, the flexible members bow radially outward, actuated by an actuator device, e.g., an actuator cable or the equivalent operatively placed in the lumen of the drive shaft and attached to the distal tapered section. A proximal force applied to the actuating device may cause the distal tapered section to move proximally, causing a bowing in the flexible members to an expanded position that varies in a directly proportional manner with the degree of proximal force applied to the actuating device up to a maximum expanded diameter that may be achieved. In this embodiment, the flexible members are biased in a relatively straightened configuration to comprise the cylindrical profile shown in FIG. 2.

In alternate embodiments, the flexible members may be biased in an outwardly bowed configuration and held in a retracted position by the catheter lumen. When the flexible members are advanced beyond the distal end of the catheter lumen, the flexible members are released and expand axially outwardly. Returning the flexible members proximally into the confines of the catheter lumen results in the flexible members retracting into a retracted position. Those skilled in the art will recognize that automatically sizes the flexible members to the blockage or to the vessel to be cleaned, without user intervention.

Prior to use, the drive shaft, the proximal mount, the flexible members and the distal mount are all contained within the catheter's lumen, and all surround the guide wire. First, a user feeds the guide wire is through the vasculature of the patient to the blockage. Next, the user advances the catheter and its contents over the guide wire to the blockage. The catheter has a generally smooth exterior and does not damage any of the blood vessels as it is advanced along the guide wire. Once the distal end of the catheter is positioned at or near the blockage, the user advances the drive shaft with respect to the catheter (and/or, equivalently, retracts the catheter with respect to the drive shaft). The axial pressure from the drive shaft pushes the abrasive nose cone out the distal end of the catheter, with the drive shaft pushing on the proximal mount, the proximal mount pushing on the flexible members, and the flexible members pushing on the distal mount. Note that the flexible members, e.g., wires, can support this relatively small amount of axial pressure, and can facilitate pushing the nose cone out of the catheter.

In the embodiment wherein the flexible members are biased in an expanded position and once the flexible members are pushed out of the catheter, the catheter wall no longer constrains them radially, and they are free to expand radially to automatically press against the sides of the occlusion without further operator intervention. A benefit of such an abrasive head is that the radial expansion of the flexible members is automatic, and does not require any additional steps from the user or any additional elements in the catheter. Furthermore, the radial expansion does not rely on centrifugal force, so that the expansion may occur at relatively low rotational speeds or at rest. The proximal mount, flexible members, distal mount and tapered distal section all rotate with the drive shaft. The tapered distal section opens, when needed, a pilot hole sufficient in diameter for the flexible members to begin to operate in gradually expanding positions, the radially expanded flexible members cut, scrape and/or grind away at the blockage in the vessel. As the blockage is abraded, the flexible members grow radially to the proper size automatically, again without any intervention from the user. Once the blockage is fully abraded, the rotation of the drive shaft is reduced or stopped, and the user retracts the drive shaft with respect to the catheter (and/or, equivalently, the user advances the catheter with respect to the drive shaft). The drive shaft pulls the proximal mount inside the catheter, the proximal mount pulls the flexible members inside the catheter and radially compresses them while doing so for removal.

In the embodiment wherein the flexible members are biased in the retracted position, proximal force applied to the actuator by the operator causes the distal tapered section to move proximally, in turn causing the flexible members to bow radially outwardly into an expanded position that is controlled by the operator's proximal force on the actuating device. Advantages of this embodiment comprise the operator's ability to actively control the diameter of the nose cone's flexible members when actuated to an expanded position. In addition, as above, centrifugal force is not used, nor is it required, to actuate the flexible members to an expanded position.

Once the blockage is fully abraded, the rotation of the drive shaft is reduced or stopped, and the user retracts the drive shaft with respect to the catheter (and/or, equivalently, the user advances the catheter with respect to the drive shaft) in accordance with the nose cone embodiments described above.

The above paragraphs are merely a summary of the disclosure, and should not be construed as limiting in any way. A more detailed description follows.

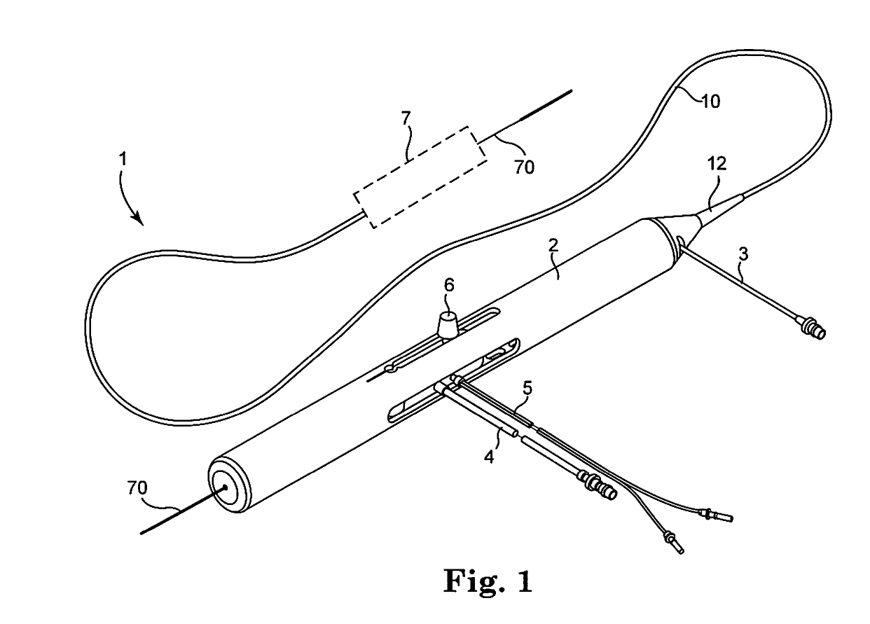

FIG. 1 is a schematic drawing of a typical rotational atherectomy device. The device includes a handle portion 2, an elongated, flexible drive shaft, and an elongated catheter 10 extending distally from the handle portion 2. The drive shaft transmits torque from a controlling proximal end 12, at or near the handle portion 2, to an abrasive head 7 at or near a distal end of the drive shaft. The catheter 10 has a lumen in which most or all of the length of the drive shaft is disposed. The drive shaft also contains an inner lumen, permitting the drive shaft to be advanced and rotated over a guide wire 70. A fluid supply line 3 may be provided for introducing a cooling and lubricating solution (typically saline or another biocompatible fluid) into the catheter 10.

The handle 2 may contain a turbine (or similar rotational drive mechanism) for rotating the drive shaft at high speeds. The handle 2 typically may be connected to a power source, such as compressed air delivered through a tube 4. A pair of fiber optic cables 5 may also be provided for monitoring the speed of rotation of the turbine and drive shaft. Details regarding such handles and associated instrumentation are well known in the industry, and are described, e.g., in U.S. Pat. No. 5,314,407, titled “Clinically practical rotational angioplasty system”, issued on May 24, 1994 to David C. Auth et al, and incorporated by reference in its entirety herein. The handle 2 also desirably includes a control knob 6 for advancing and retracting the turbine and drive shaft with respect to the catheter 10 and the body of the handle.

There are several ways to rotate the drive shaft during operation. In most cases, a motor is attached to the drive shaft at or near the proximal end of the drive shaft. A suitable control system for such a motor is disclosed in U.S. patent application Ser. No. 10/272,126, to Shturman et al, titled “Control system for rotational angioplasty device”, and published on Jun. 26, 2003 as U.S. Patent Application Publication No. US 2003/0120296 A1, which is incorporated by reference herein in its entirety.

Other methods of drive shaft rotation are possible as well. For instance, the user can directly rotate the drive shaft by hand. As another example, the user can turn a crank connected to the drive shaft. As a further example, the user can turn a crank, with the crank driving a geared system that scales up the rotational speed of the drive shaft by a particular factor over the rotational speed of the crank. Other suitable rotation-producing systems are possible as well.

The present application is directed mainly to a design of the abrasive head 7. In this respect, many or all of the other elements of the known atherectomy device of FIG. 1 may be used with the present disclosed head design, including the catheter 10, the guide wire 70, and the handle 2 along with its controls and its inputs and outputs.

Note that FIG. 1 shows the guide wire 70 extending beyond the abrasive head 7. Typically, the guide wire 70 is the first element of the atherectomy device 1 inserted into the blood vessel. Being much thinner than the catheter 10, the guide wire 70 is much easier to navigate through the vasculature of the patient from the insertion point to the blockage. Once the guide wire 70 reaches the blockage, the catheter 10 may be advanced along the guide wire 70 until the abrasive head 7 is suitably located at or near the blockage.

Once the abrasive head 7 is placed, there are several options available for the guide wire. In most cases considered herein, the guide wire 70 is left in place and extends beyond the distal end of the abrasive head 7. This helps provide stability for the distal mount during use. In other cases, the guide wire 70 may be retracted partially into the catheter 10, so that it does not extend into or beyond the abrasive head. In still other cases, the guide wire 70 may be retracted completely from the catheter 10. After the blockage has been removed, retracting the catheter 10 does not require the use of a guide wire 70, since the retraction involves pulling the catheter 10 from the insertion point and does not require any particular navigation through the vasculature of the patient.

FIGS. 2 and 3 show an exemplary abrasive nose cone 7A, operatively attached to the distal end of the drive shaft 10 which is disposed, axially translatable and rotatable within catheter 20. The abrasive nose cone 7A comprises a plurality of flexible, elongate members 100, which can be wires or strips of suitable cutting or grinding material. The wires may have a circular cross-section, or may have an asymmetric cross section that may enhance their cutting or scraping abilities. For instance, an asymmetric cross-section may be a half-circle, triangular, rectangular, square, and/or may include one or more corners. In FIG. 2, the flexible members 100 are radially compressed and axially extended, as they are when inside the lumen of catheter 100 and in a retracted position. In FIG. 3, the flexible members 100 are axially compressed and radially expanded, as they are when outside the catheter and during use in an expanded position.

As illustrated, the proximal ends of the members 100 are all attached to a proximal mount 40. In some cases, some or all of the members 100 may attach to each other before attaching to the proximal mount 40. In other cases, the members 100 may attach only to the proximal mount 40 and do not attach to each other. The proximal mount 40 is mechanically and rotatably coupled to the drive shaft 10 in most cases being at or near the distal end of the drive shaft. As the drive shaft 10 rotates, the proximal mount 40 rotates as well. Similarly, if the drive shaft 10 is axially translated within the catheter 20, the proximal mount 40 follows. The proximal mount 40 may be made integral with the drive shaft 10, or may be made separately from the drive shaft 10 and attached to the drive shaft 10. In many cases, the drive shaft 10 is formed as one or more helically coiled wires, and the proximal mount 40 is a solid structure operatively attached to the distal end of the drive shaft 10.

In the same manner that the proximal ends of the flexible members 100 are attached to a common proximal mount 40, the distal ends of the flexible members are attached to a common distal mount 50 which is operatively attached to, or integral with, the distal tapered section 102. In some cases, some or all of the members 100 may attach to each other before attaching to the distal mount 50 which is operatively attached to, or integral with, the distal tapered section 102. In other cases, the members 100 may attach only to the distal mount 50 and do not attach to each other. In all cases, the distal tapered section 102 is connected with the drive shaft 10 via the distal mount 50, the flexible members 30A and the proximal mount 40. Therefore, the distal tapered section 102 translates and rotates in concert with the drive shaft 10, the flexible members 100, and the proximal and distal mounts 40, 50.

In many cases, the guide wire 70 passes through the proximal and distal mounts 40, 50 and through a lumen 45 (shown in FIG. 3) within the flexible members 100 and keeps the proximal and distal mounts 40, 50 and the flexible members 100, in certain embodiments, roughly centered along the rotational axis of the drive shaft 10 as the drive shaft 10 rotates. Such stability may be useful at the high rotational speeds, e.g., 20,000 rpm or higher, that are generally required for rotational atherectomy.

As shown in FIG. 3, the flexible members 100 comprise an expanded elliptical profile wherein each flexible member 100 is arranged circumferentially and angularly equidistant from the adjacent flexible member 100. This arrangement results in a symmetric plurality of flexible members 100 with a center of mass on the axis of rotation of the drive shaft. Accordingly, high-speed rotation of the expanded flexible members 100 will achieve a rotational (and abrasive/cutting) diameter that is equivalent to the resting diameter of the expanded flexible members.

Alternate embodiments may comprise the flexible members 100 having an at least partially elliptical profile wherein a portion of the plurality of the flexible members 100 comprise an elliptical profile as described above while the remainder of the flexible members 100 are arranged with larger, or smaller, distances from one flexible member 100 to the next adjacent flexible member 100. This alternative arrangement results in an asymmetric plurality of flexible members 100 with a center of mass for the flexible members that is not on the drive shaft 10 axis of rotation. As a result, high-speed rotation of the abrasive nose cone will result in an eccentric, or orbital, motion as is well described in U.S. Pat. No. 6,494,890 to Shturman and is incorporated by reference in its entirety herein. The advantage of an eccentric, or asymmetric, plurality of flexible members 100 is that the rotational (and abrasive/cutting) diameter of the expanded plurality of flexible members 100 is larger than the resting diameter of the expanded plurality of flexible members 100. This, in turn, allows a lower profile abrasive element, i.e., abrasive nose cone 7A, to be inserted into the patient's vasculature which, inter alia, reduces trauma.

Alternative mechanisms may be employed to achieve offset of the center of mass from the rotational axis of the drive shaft. For example, at least part of one or more of the plurality of flexible members 100 may comprise a material that is of a density that is more, or less, dense than the density of the remaining flexible members 100. Those skilled in the art will now recognize the utility in such an arrangement, as well as the large number of permutations that may be engineered into the present invention. Each such resulting embodiment is well within the scope of the present invention.

The distal tapered section 102 is located distal to, and adjacent, the flexible members 100 and connected thereto by distal mount 50 as described above. The distal tapered section 102 comprises a proximal fixed cylindrical profile and a distal fixed conical profile. The distal tapered section 102 further comprises a lumen therethrough (not shown) to allow axial translation over the guide wire 70. Since the distal mount 50 is operatively attached to, or integral with, the distal tapered section 102, the distal mount 50 is also free to axially translate, or slide, along the guide wire 70.

The exterior surface of the flexible members 100 and the distal tapered section 102 may be coated, in whole or in part(s), with an abrasive material. The abrasive material may be any suitable material, such as diamond powder, fused silica, titanium nitride, tungsten carbide, aluminum oxide, boron carbide, or other ceramic materials. In some cases, the abrasive material includes diamond chips or diamond dust particles, attached directly to the exterior of the flexible members 100 and the distal tapered section 102 by a suitable binder. The material may be attached using well known techniques, such as conventional electroplating or fusion technologies (see, for example, U.S. Pat. No. 4,018,576, which is incorporated by reference in its entirety herein). Alternately, the exterior surface of the flexible members 100 and/or the distal tapered section 102 may be mechanically or chemically roughened, and/or may be etched or cut, as with a laser, to provide a sanding, grinding, cutting, or slicing surface.

The flexible members 100 comprise sharp cutting side edges 104 along both sides to provide a cutting surface. These cutting side edges 104 are exposed only when the nose cone is in an expanded position as in FIG. 2. Otherwise, when in a retracted position as in FIG. 1, the cutting edges 104 are not exposed.

Thus, the nose cone allows for grinding of occluding material along abrasive surface of the distal tapered section 102 and the retracted flexible members 100. In addition, the expanded flexible members 100 allow for cutting of occluding material.

As provided in the Figures, a suction means as is well known in the art may be provided to enable a debris flow proximally through the lumen of the drive shaft and away from the occlusion. Alternatively, a distal protection device, also well known in the art, may be employed to capture the debris generated during the atherectomy method of the present invention.

The description of the invention and its applications as set forth herein is illustrative and is not intended to limit the scope of the invention. Variations and modifications of the embodiments disclosed herein are possible and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

We claim:

1. A rotational atherectomy apparatus for abrading tissue, comprising:
a flexible, elongated, rotatable drive shaft having a proximal end and a distal end opposite the proximal end;
an abrasive nose cone comprising:
a plurality of elongate, flexible members proximate the distal end of the drive shaft, each member in the plurality being fixed at both a proximal end and a distal end opposite the proximal end and comprising a biased retracted position having a cylindrical profile, an exterior surface having abrasive coating thereon, and sharp cutting side edges, wherein the elongate, flexible members are rotatable with the drive shaft;
a proximal mount operatively connected with and rotatable with the drive shaft and fixedly connected to the proximal ends of all the flexible members in the plurality; and
a distal mount axially separated from the proximal mount and fixedly connected to the distal ends of all the flexible members in the plurality and rotatable with the drive shaft and the plurality of elongate, flexible members; and
a distal tapered section comprising a proximal fixed cylindrical profile and a distal fixed conical profile, and a lumen therethrough, the distal tapered section attached to the distal mount and rotatable with the drive shaft and the plurality of elongate, flexible members;
wherein when the axial separation of the proximal and distal mounts is reduced, each member in the plurality bows outward from the drive shaft and expands radially away from the biased retracted position to an expanded position.

2. The rotational atherectomy apparatus of claim 1, further comprising an actuating device which allows the operator to control the reduction of axial separation of the proximal and distal mounts.

3. The rotational atherectomy apparatus of claim 1, wherein the flexible members are wires.

4. The rotational atherectomy apparatus of claim 3, wherein at least one of the wires has a circular cross-section.

5. The rotational atherectomy apparatus of claim 3, wherein at least one of the wires has a non-circular cross-section.

6. The rotational atherectomy apparatus of claim 1, wherein the flexible members are mounted in a parallel array and are spaced angularly apart from each other.

7. A rotational atherectomy apparatus for abrading occluding material within the lumen of a blood vessel, comprising:
a flexible, elongated, rotatable drive shaft having a proximal end and a distal end opposite the proximal end;
an abrasive nose cone comprising:
a plurality of elongate, flexible members proximate the distal end of the drive shaft, each member in the plurality being fixed at both a proximal end and a distal end opposite the proximal end and comprising a biased retracted position having an at least partially elliptical profile, an exterior surface having abrasive coating thereon, and sharp cutting side edges, wherein the elongate, flexible members are rotatable with the drive shaft;
a proximal mount operatively connected with and rotatable with the drive shaft and fixedly connected to the proximal ends of all the flexible members in the plurality; and
a distal mount axially separated from the proximal mount and fixedly connected to the distal ends of all the flexible members in the plurality and rotatable with the drive shaft and the plurality of elongate, flexible members; and
a distal tapered section comprising a proximal fixed cylindrical profile and a distal fixed conical profile, and a lumen therethrough, the distal tapered section attached to the distal mount and rotatable with the drive shaft and the plurality of elongate, flexible members;
wherein when the axial separation of the proximal and distal mounts is reduced, each member in the plurality bows outward from the drive shaft and expands radially away from the biased retracted position to an expanded position.

8. The rotational atherectomy apparatus of claim 7, wherein the plurality of flexible members comprises flexible members are arranged asymmetrically.

9. The rotational atherectomy apparatus of claim 7, wherein the flexible members are wires.

10. The rotational atherectomy apparatus of claim 9, wherein at least one of the wires has a circular cross-section.

11. The rotational atherectomy apparatus of claim 9, wherein at least one of the wires has a non-circular cross-section.

12. The rotational atherectomy apparatus of claim 7, the drive shaft having an axis of rotation and the plurality of flexible members further comprising a center of mass located on the axis of rotation.

13. The rotational atherectomy apparatus of claim 7, the drive shaft having an axis of rotation and the plurality of flexible members further comprising a center of mass offset from the axis of rotation.

14. A rotational atherectomy apparatus for abrading occluding material within the lumen of a blood vessel, comprising:
a flexible, elongated, rotatable drive shaft having a proximal end and a distal end opposite the proximal end;
an abrasive nose cone comprising:
a plurality of elongate, flexible members proximate the distal end of the drive shaft, each member in the plurality being fixed at both a proximal end and a distal end opposite the proximal end and comprising a biased expanded position having an at least partially elliptical profile, an exterior surface having abrasive coating thereon, and sharp cutting side edges, wherein the elongate, flexible members are rotatable with the drive shaft;
a proximal mount operatively connected with and rotatable with the drive shaft and fixedly connected to the proximal ends of all the flexible members in the plurality, each flexible member comprising at least one material having a density, wherein the density of the plurality of flexible members is arranged asymmetrically; and
a distal mount axially separated from the proximal mount and fixedly connected to the distal ends of all the flexible members in the plurality and rotatable with the drive shaft and the plurality of elongate, flexible members; and
a distal tapered section comprising a proximal fixed cylindrical profile and a distal fixed conical profile, and a lumen therethrough, the distal tapered section attached to the distal mount and rotatable with the drive shaft and the plurality of elongate, flexible members;
wherein the axial separation of the proximal and distal mounts automatically changes to match the lumen of the blood vessel, each member in the plurality bows outward from the drive shaft and expands radially to the biased expanded position.

15. The rotational atherectomy apparatus of claim 14, wherein the plurality of flexible members comprises flexible members are arranged asymmetrically.

16. The rotational atherectomy apparatus of claim 14, wherein the flexible members are wires.

17. The rotational atherectomy apparatus of claim 16, wherein at least one of the wires has a circular cross-section.

18. The rotational atherectomy apparatus of claim 16, wherein at least one of the wires has a non-circular cross-section.

19. The rotational atherectomy apparatus of claim 14, the drive shaft having an axis of rotation and the plurality of flexible members further comprising a center of mass located on the axis of rotation.

20. The rotational atherectomy apparatus of claim 14, the drive shaft having an axis of rotation and the plurality of flexible members further comprising a center of mass offset from the axis of rotation.

21. A system for abrading an occlusion in a blood vessel, comprising:
a guide wire for negotiating a vasculature of a patient to the blockage;
a catheter advanceable over the guide wire;
a flexible, elongated, drive shaft rotatable within the catheter and advanceable over the guide wire, the drive shaft having a proximal end remaining external to the vasculature of the patient and a distal end opposite the proximal end;
means for rotating the drive shaft; and
an abrasive nose cone comprising:
a plurality of elongate, flexible members proximate the distal end of the drive shaft, each member in the plurality being fixed at both a proximal end and a distal end opposite the proximal end and comprising a biased retracted position having an at least partially elliptical profile, an exterior surface having abrasive coating thereon, and sharp cutting side edges, wherein the elongate, flexible members are rotatable with the drive shaft;
a proximal mount operatively connected with and rotatable with the drive shaft and fixedly connected to the proximal ends of all the flexible members in the plurality; and
a distal mount axially separated from the proximal mount and fixedly connected to the distal ends of all the flexible members in the plurality and rotatable with the drive shaft and the plurality of elongate, flexible members; and
a distal tapered section comprising a proximal fixed cylindrical profile and a distal fixed conical profile, and a lumen therethrough, the distal tapered section attached to the distal mount and rotatable with the drive shaft and the plurality of elongate, flexible members;
wherein when the axial separation of the proximal and distal mounts is reduced, each member in the plurality bows outward from the drive shaft and expands radially away from the biased retracted position to an expanded position.

22. A method for abrading an occlusion in a blood vessel, comprising:
advancing a guide wire through a vasculature of a patient to the blockage;
advancing a catheter over the guide wire to the blockage;
advancing a rotatable drive shaft within the catheter to the blockage, the drive shaft having an abrasive nose cone operatively connected thereto and comprising:
a plurality of elongate, flexible members proximate the distal end of the drive shaft, each member in the plurality being fixed at both a proximal end and a distal end opposite the proximal end and comprising a biased retracted position having an at least partially elliptical profile, an exterior surface having abrasive coating thereon, and sharp cutting side edges, wherein the elongate, flexible members are rotatable with the drive shaft;
a proximal mount operatively connected with and rotatable with the drive shaft and fixedly connected to the proximal ends of all the flexible members in the plurality; and
a distal mount axially separated from the proximal mount and fixedly connected to the distal ends of all the flexible members in the plurality and rotatable with the drive shaft and the plurality of elongate, flexible members; and
a distal tapered section comprising a proximal fixed cylindrical profile and a distal fixed conical profile, and a lumen therethrough, the distal tapered section attached to the distal mount and rotatable with the drive shaft and the plurality of elongate, flexible members;
creating a pilot hole through the occlusion with the tapered distal section;
actuating an actuating device to achieve a proximal force on the distal tapered section;
reducing the axial separation of the proximal and distal mounts in response to the proximal force;
bowing the plurality of flexible members radially outward from the drive shaft;
rotating the drive shaft;
abrading the occlusion through repeated contact with the plurality of bowed flexible members;
stopping the rotation of the drive shaft;
withdrawing the plurality of flexible members into the catheter, the flexible members becoming radially compressed upon withdrawal into the catheter; and
withdrawing the catheter, the plurality of flexible members, the proximal and distal mounts and the drive shaft with abrasive nose cone from the vasculature of the patient.
